(19) United States
(12) Patent Application Publication
(10) Pub. No.: US 2025/0359953 A1
(43) Pub. Date: Nov. 27, 2025

(54) CLOSED-LOOP FEEDBACK BASED ON MIXED DIMENSIONALITY IMAGING

(71) Applicant: Intuitive Surgical Operations, Inc., Sunnyvale, CA (US)

(72) Inventors: Samuel B. Schorr, Sunnyvale, CA (US); Shibing Liu, Sunnyvale, CA (US); Troy K. Adebar, San Jose, CA (US)

(73) Assignee: Intuitive Surgical Operations, Inc., Sunnyvale, CA (US)

(21) Appl. No.: 19/214,681

(22) Filed: May 21, 2025

Related U.S. Application Data
(60) Provisional application No. 63/650,803, filed on May 22, 2024.

(51) Int. Cl. A61B 34/32; A61B 34/00; A61B 34/20; A61B 34/30; A61B 90/00; G16H 40/63

(52) U.S. Cl. CPC A61B 34/32; A61B 34/20; A61B 34/25; A61B 34/70; A61B 90/37; G16H 40/63; A61B 2034/2065; A61B 2034/301; A61B 2090/3762; A61B 2090/378

(57) ABSTRACT

A medical system includes a manipulator assembly configured to drive a flexible elongate device, and a control system coupled to the manipulator assembly. The control system is configured to receive a three-dimensional (3D) image of a distal portion of the flexible elongate device and a target structure, determine, based on the 3D image, a two-dimensional (2D) imaging plane for viewing movement of the distal portion of the flexible elongate device from a first position captured in the 3D image to a second position that points toward the target structure, receive 2D images in the 2D imaging plane captured over time, and control the manipulator assembly to move the distal portion of the flexible elongate device from the first position to the second position based on the 2D images.

### CROSS-REFERENCE TO RELATED APPLICATIONS

This application claims the benefit of priority under 35 U S.C. § 119 (e) to U.S. Provisional Patent Application Ser. No. 63/650,803, filed on May 22, 2024, which is hereby incorporated by reference herein in its entirety.

### FIELD

Disclosed embodiments relate to improved robotic and/or medical (including surgical) devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, that can be inserted into anatomic passageways and navigated toward a region of interest within the patient anatomy.

### SUMMARY

The following presents a simplified summary of various examples described herein and is not intended to identify key or critical elements or to delineate the scope of the claims.

In some examples, embodiments of the disclosure relate to a manipulator assembly configured to drive a flexible elongate device; and a control system coupled to the manipulator assembly, the control system configured to: receive a three-dimensional (3D) image of a distal portion of the flexible elongate device and a target structure; determine, based on the 3D image, a two-dimensional (2D) imaging plane for viewing movement of the distal portion of the flexible elongate device from a first position captured in the 3D image to a second position that points toward the target structure; receive 2D images in the 2D imaging plane captured over time; and control the manipulator assembly to move the distal portion of the flexible elongate device from the first position to the second position based on the 2D images.

In some examples, embodiments of the disclosure relate to a non-transitory machine-readable medium comprising a plurality of machine-readable instructions executed by one or more processors associated with a medical system, the plurality of machine-readable instructions causing the one or more processors to perform a method comprising: receiving a three-dimensional (3D) image of a distal portion of a flexible elongate device configured to be driven by a manipulator assembly, and a target structure; determining, based on the 3D image, a two-dimensional (2D) imaging plane for viewing movement of the distal portion of the flexible elongate device from a first position captured in the 3D image to a second position that points toward the target structure; receiving 2D images in the 2D imaging plane captured over time; and controlling the manipulator assembly to move the distal portion of the flexible elongate device from the first position to the second position based on the 2D images.

In some examples, embodiments of the disclosure relate to a method for operating a medical system, comprising: receiving a three-dimensional (3D) image of a distal portion of a flexible elongate device configured to be driven by a manipulator assembly, and a target structure; determining, based on the 3D image, a two-dimensional (2D) imaging plane for viewing movement of the distal portion of the flexible elongate device from a first position captured in the 3D image to a second position that points toward the target structure; receiving 2D images in the 2D imaging plane captured over time; and controlling the manipulator assembly to move the distal portion of the flexible elongate device from the first position to the second position based on the 2D images.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4 is a simplified diagram of a medical system including an imaging device according to some embodiments.

FIG. 5 depicts a 3D image of an anatomical structure, a flexible elongate device, and a spatial relationship between the anatomical structure and the elongate device, respectively, according to some embodiments.

FIG. 6 depicts a 3D representation of a flexible elongate device and projections of the flexible elongate device onto select 2D imaging planes according to some embodiments.

FIGS. 7A, 7B, and 7C depict an example targeting operation according to some embodiments.

FIG. 8 is a flowchart of a method according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. A medical system that uses flexible elongate devices (e.g., catheters, bronchoscopes, endoscopes, etc.) can be used to move a flexible elongate device, or a portion of a flexible elongate device; including a medical tool that may be enclosed by the flexible elongate device. Herein, and as will be described in greater detail later in the instant disclosure, “movement” of a flexible elongate device can be further categorized as “navigating” (or navigation) and “targeting” (or “aiming”). Navigating may refer to the movement of the flexible elongate device to a target region. For example, during a diagnostic or therapeutic procedure such as bronchoscopy, a flexible elongate device may be inserted through a naturally or surgically created anatomic orifice of a patient (e.g., nose, mouth, tracheostomy) through the tracheobronchial tree to a target region (e.g., region containing or proximate to a peripheral pulmonary lesion (PPL)). Navigating can include articulation of the flexible elongate device at any portion along the length of the flexible elongate device. Further, navigating can be associated with bulk movement of the flexible elongate device relative to a so-called insertion axis of the medical system controlling the movement of the flexible elongate device. In contrast, targeting (or aiming) generally refers to articulation of the distal portion of the flexible elongate device to aim (or point) the flexible elongate device at a specified target or target structure in the target region. Embodiments of this disclosure are applicable to both navigating and targeting. As such, changes in a flexible elongate device from a first position to a second position (or, more generally from a first pose or shape to a second pose or shape) are described as “movements” (including other references such as “move” and “to move”). Further, a movement may be composed of an ordered sequence of movements. For example, a flexible elongate device can be moved from a first shape to a second shape while passing through any number of intermediate shapes, where changes between intermediate shapes can be described as movements.

A more detailed discussion of the medical system, a medical instrument including a flexible elongate device, and methods for navigating and targeting using mixed dimensionality imaging, including resulting benefits, is provided below in reference to the figures.

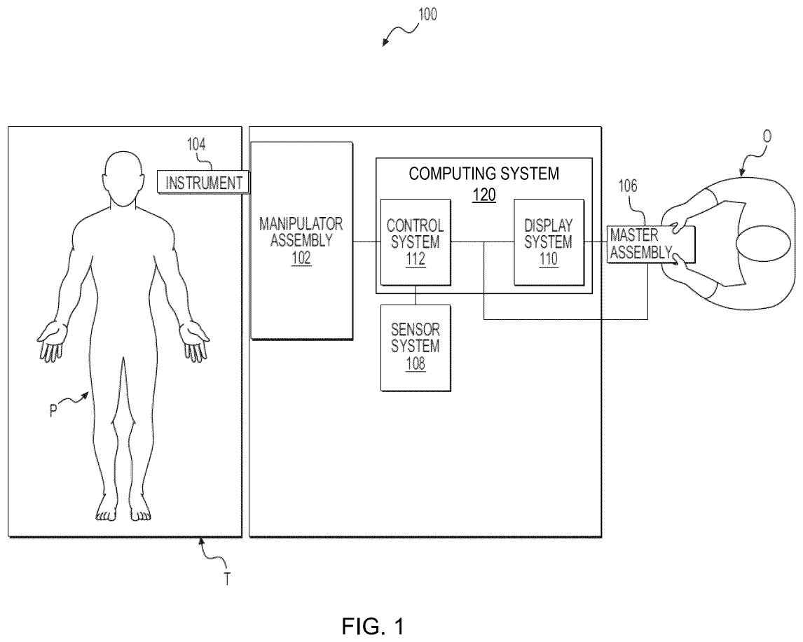

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models. In some instances, and as described below, intra-operative images can be used to locate and map (or track) the location of the medical instrument 104 as the medical instrument 104 moves through the patient anatomy.

In some examples, for purposes of image-guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, where different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigating and/or targeting assistance to operator O when controlling the medical instrument 104 during an image-guided medical procedure. In general, navigating and targeting sequences can be visualized using the visualization system based on an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

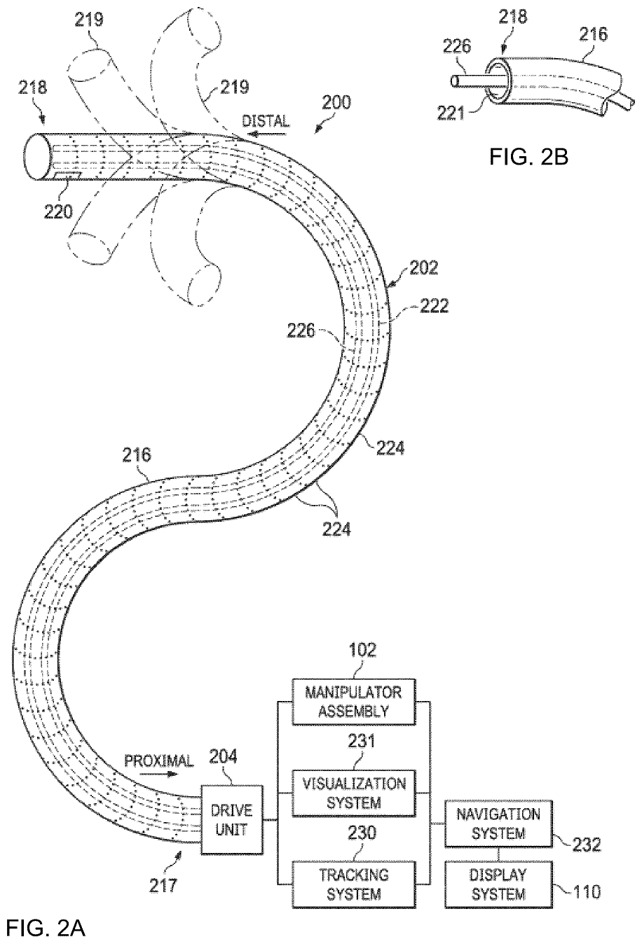

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexible body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

Embodiments of the instant disclosure are not reliant on a shape sensor, position sensor, or historical position and/or pose data to reconstruct the shape of the flexible body 216. In one or more embodiments, at least one three-dimensional intra-operative image is acquired (e.g., using cone beam computed tomography (CBCT)) and used to determine, at least, a position and orientation of the flexible body 216, or a portion of the flexible body 216, relative to patient anatomy. Subsequent tracking and visualization of the elongate device 202 can be performed using additional two- or three-dimensional intra-operative images. For example, to perform a targeting operation a three-dimensional image of a distal portion of the flexible elongate device 202 and a target structure (e.g., PPL) can be acquired. The three-dimensional image may be used to locate and visualize the flexible elongate device 202 within the patient anatomy. Then, based on the three-dimensional image data, a two-dimensional imaging plane for viewing movement of the distal portion of the flexible elongate device 202 from a first position to a second position is determined, where the second position points toward the target structure. Movement of the distal portion of the flexible elongate device 202 from the first position to the second position is monitored, validated, and visualized using one or more 2D images in the 2D imaging plane captured over time. A detailed description is provided below.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical tool 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

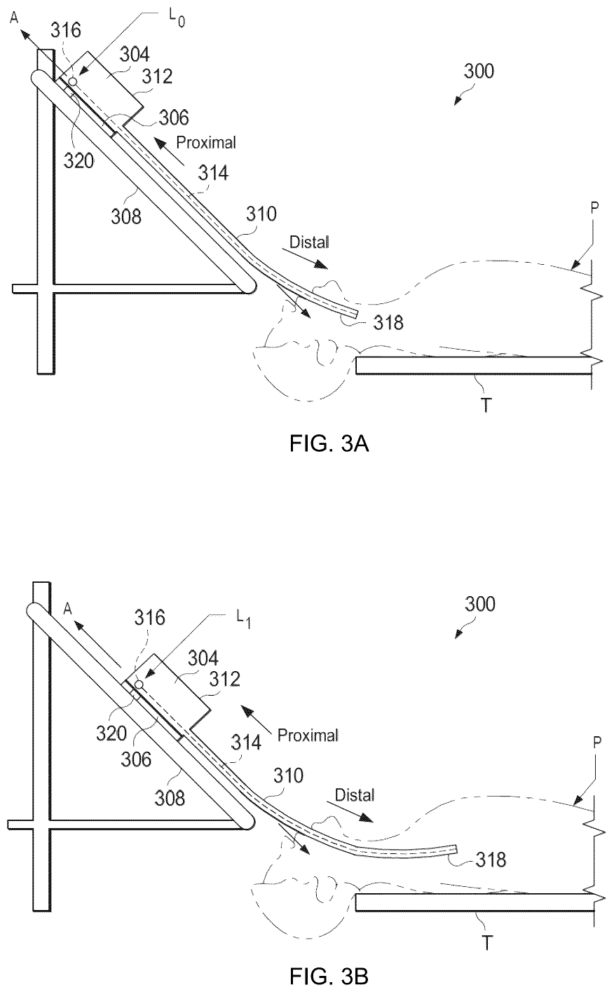

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

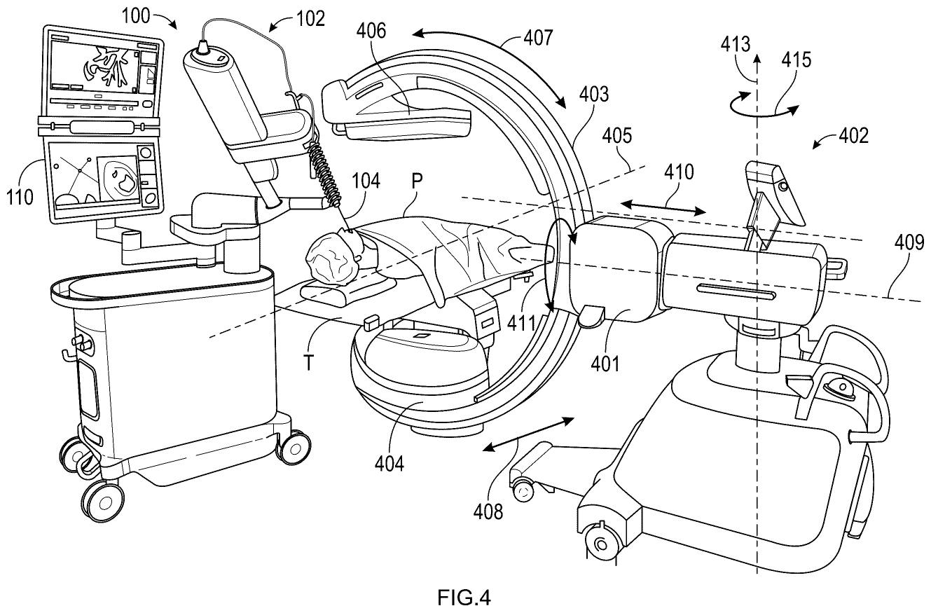

FIG. 4 depicts another view of a medical system 100 with an imaging system 402. As discussed with reference to FIG. 1, the medical system 100 may include a manipulator assembly 102 that controls the operation of a medical instrument 104 in performing various procedures on a patient P. In FIG. 4, patient P is on a table T. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

In accordance with some embodiments, the imaging system 402, which may include more than one imaging device, is cable of acquiring images of more than one dimensionality. For example, in some embodiments, the imaging system 402 implements cone beam computed tomography (CBCT) and fluoroscopy to acquire three-dimensional (3D) and two-dimensional (2D) images, respectively. In other examples, two and a half-dimensional (2.5D) images (or scans) e.g., sectional images that are reconstructed into a 3D volume, may be obtained, for example, using tomosynthesis imaging. In such a case, the imaging system 402 can be used to acquire 2.5D and 2D images. The imaging system 402 can use a combination of imaging devices such as a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device for capturing images, or use any of the aforementioned imaging devices in different modes to acquire images (e.g., of the distal end of medical instrument 104) of mixed dimensionalities including at least a 2D image (e.g., 2D and 3D, 2D and 2.5D).

In general, the frame rate or temporal resolution of images acquired by the imaging system 402 may be related to the dimensionality of the acquired image. Typically, the temporal resolution of 2D images is greater than that of 2.5D images and the temporal resolution of 2.5D images is greater than 3D images. For example, in instances where a 3D image (or scan) is acquired using CBCT or other 3D imaging modality, the acquired 3D images (or scans) may have a limited temporal resolution of, for example, no better than 0.1 Hz. In contrast, 2.5D and 2D images (or scans) may have considerably higher temporal resolution than their 3D-imaging counterparts. For example, fluoroscopy or other 2D imaging device may have a temporal resolution greater than 10 Hz making these devices and techniques suitable for real-time (or near real-time) use (e.g., structuring and displaying the acquired images sequentially according to acquisition time to form a video).

Embodiments disclosed herein are readily applicable to an imaging system 402 capable of acquiring images (or scans) of mixed dimensionality including at least a 2D image (e.g., 2D and 3D, 2D and 2.5D), whether the imaging system 402 is composed of more than one imaging device or can be considered a single imaging device operable in different modes or imaging modalities.

In the example of FIG. 4, the depicted imaging system 402 is a mobile C-arm style device that can implement fluoroscopy and CBCT to acquire 2D and 3D images (or scans), respectively. The depicted imaging system 402, being mobile, can be translated along a floor. FIG. 4 depicts the translational movement of the imaging system 402 using a first translational degreed of freedom (DOF) 408 and a second translational DOF 410, where the first and second translation DOFs 408, 410 are orthogonal to each other and coplanar with the floor. In general, the translational motion of the imaging system 402 with respect to the floor need not be defined using orthogonal degrees of freedom or an orthogonal coordinate system (e.g., Cartesian). Further, the depicted imaging system 402, by nature of its mobility, can be rotated about itself. For example, a vertical axis 413 (i.e., orthogonal to a plane defined by the floor) may be placed at an arbitrary location (e.g., the center of mass of the imaging system 402) to define a first rotational axis 413 about which the imaging system 402, itself, may be rotated over a first rotational DOF 415. While the imaging system 402 depicted in FIG. 4 is mobile, non-mobile imaging systems 402 may be used without limitation. For example, in some implementations, the imaging system 402, or components of the imaging system 402, can be disposed on a rail system to achieve one or more of the aforementioned degrees of freedom of the imaging system 402. For example, the imaging system 402 may be suspended using a ceiling mounted rail system to allow motion of the imaging device along the first translational DOF 408. In some implementations still, the imaging system 402 can include an extendable boom or rotational joint to realize the second translational DOF 410 and the first rotational DOF 415, respectively. In other implementations, the imaging system 402 is fixed or unmovable in the aforementioned degrees of freedom. In some instances, and as described below, one or more degrees of freedom, for example, the first translational degree of freedom 408, is provided using the table T, where the table T moves relative to a fixed imaging system 402.

Continuing with the example of FIG. 4, the depicted imaging system 402 includes a gantry 401 about which a C-arm 403 is suspended or attached. In some implementations, the gantry 401 defines a second rotational axis 409 and includes a rotational joint to provide rotation of the C-arm 403 over a second rotational DOF 411. The C-arm 403 includes an X-ray generation system 404 and an X-ray collection system 406. The X-ray generation system 404 generates an X-ray beam that is passed through the patient P and collected by the X-ray collection system 406. A detailed description of the inner elements of the X-ray generation system 404 and X-ray collection system 406 exceed the scope of this disclosure. However, in general, an X-ray generation system 404 includes an X-ray tube to generate the X-ray beam, one or more filters to tailor the flux of the generated X-ray beam, and a collimator to direct and shape the X-ray beam. Similarly, in general, an X-ray collection system 406 includes an anti-scatter grid, a detector (e.g., flat panel detector, image intensifiers, etc.), and a dosage meter to measure and validate the received flux.

In one or more implementations, the C-arm 403 of the imaging system 402 defines a third rotational axis 405 that is coaxial with the center of a virtual circle that follows the contours of the C-arm. The C-arm 403, or elements disposed on the C-arm 403 such as the X-ray generation system 404 and the X-ray collection system 406, can be rotated about the third rotational axis 405 over a third rotational DOF 407. In the example of FIG. 4, the imaging system 402 is positioned such that the third rotational axis 405 is approximately aligned with the spine of the patient P. In other instances, the imaging system 402 or patient P may be positioned such that the third rotational axis 405 is substantially coplanar with the patient P (e.g., coplanar with the spine of the patient P) but not aligned with spine of the patient P. For example, the third rotational axis 405 can be coplanar and perpendicular to the spine of the patient P. In this case, consider an imaging system 402 like that depicted FIG. 4 positioned at the foot of the table T (i.e., near the feet of the patient P).

The imaging system 402 can further include an image processing unit (not depicted) that processes the collected X-ray beam, collected as X-ray data, to form an image. Depending on the operation of the imaging system 402, the resulting image may be 2D (e.g., fluoroscopy), 3D (e.g., CBCT), or some other dimensionality (e.g., 4D, with consideration of temporal data). The imaging system 402 can further include a dedicated control system, that is, an imaging control system to control aspects of the imaging system 402 such as manipulation of the imaging system 402 through one or more of its degrees of freedom, the initiation and termination of an X-ray beam, and the flux of a generated X-ray beam. In some implementations, control of the imaging system 402 is performed by, or communicably coupled to, the control system 112 of the overarching medical system 100.

In some implementations, table T can provide one or more degrees of freedom. Degrees of freedom provided by table T may have the same relative effect with respect to the positioning and orientation of patient P relative to an imaging system 402, or elements of an imaging system (e.g., X-ray generation system 404 and X-ray collection system 406), as the degrees of freedom previously described with respect to imaging system 402. In some instances, degrees of freedom of the table T are used to position and orient patient P relative to the imaging system 402 in the absence of degrees of freedom provided by the imaging system 402 itself. For example, an imaging system 402 may be fixed with respect to one or more of the first and second translational degrees of freedom (408, 410). In these instances, table T may be translatable (e.g., using a rail system) to position the patient in a translational plane defined by the first and second translational DOFs (408, 410). In other instances, degrees of freedom provided by table T may be redundant in view of those provided by the imaging system 402 or may be used to extend the range of motion of one or more degrees of freedom provided by the imaging system 402. Table T can define a longitudinal axis (not depicted) that extends along the length of the table T, or from the “head-to-toe” of a patient P lying prone or supine on the table T. Similarly, table T can define a transverse axis (not depicted) that extends along the width of the table T, or from “shoulder-to-shoulder” of a patient P lying prone or supine on the table T. In some implementations, table T is rotatable about the transverse axis, where rotation of table T relative to the transverse axis raises or lowers the head or feet of patient P. That is, rotation of the table T relative to the transverse axis may be used to position a patient P that is lying prone or supine on the table T into the so-called Trendelenburg and reverse Trendelenburg positions. Likewise, in some implementations, table T is rotatable about the longitudinal axis, where rotation of table T relative to the longitudinal axis may tilt the right or left side of a patient P lying prone or supine on the table T up and down.

As depicted in FIG. 4, the medical system 100 may include a display system 110 for displaying an image or representation of the procedural site and the medical instrument 104. For example, the display system 110 can display an image representative of an anatomical structure and an inserted medical instrument 104, the image acquired using the imaging system 402. In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional (2D), two and a half-dimensional (2.5D), three-dimensional (3D), or higher-dimensional (e.g., including, for example, time based or velocity-based information) images. In some examples, one or more models are created from pre-operative or intra-operative image data sets and the virtual images are generated using the one or more models. In some instances, and as described below, intra-operative images can be used to locate and map (or track) the location of the medical instrument 104 as the medical instrument 104 moves through the patient anatomy.

The control system 112 of the medical system 100 may include a virtual visualization system that makes use of images acquired by the imaging system 402 to provide navigating and/or targeting assistance to operator O when controlling the medical instrument 104 during an image-guided medical procedure. In general, navigating and targeting sequences can be visualized using the visualization system based on an acquired pre-operative or intra-operative dataset of anatomic passageways of the patient P. The control system 112 or a separate computing device may convert images acquired with the imaging system 402, using programmed instructions alone or in combination with operator inputs, into a model of the patient anatomy. The model may include a segmented 2D, 2.5D, or 3D composite representation of a partial or an entire anatomic organ or anatomic region.

As will be described below, navigating and/or targeting can be implemented automatically (i.e., without direct human intervention) by the control system 112, in accordance with some embodiments. In some embodiments, navigating and/or targeting is performed by operator O through either manual manipulation of either the medical instrument 104 or manipulator assembly 102, or through use of the master assembly (106) (e.g., teleoperated, non-teleoperated). In some embodiments, a hybrid of automated actions and operator control is used when controlling the medical instrument 104 for navigating and/or targeting. For example, operator O may supervise automated control operations, select automated control operations, approve of proposed automated control operations, or any combination thereof. In accordance with some embodiments, and regardless of the nature of control (e.g., fully automated, operator controlled, etc.), navigating and targeting assistance is provided through, at least, the acquisition of real-time (or near real-time) 2D images of the procedure site, where the 2D images are acquired according to a projection that optimizes visualization of the movement (navigating or targeting) of the medical instrument 104.

With respect to the acquisition of 2D images using the imaging system 402, a 2D image is defined relative to a 2D imaging plane. Using the imaging system 402 of FIG. 4 as an example, the 2D imaging plane may be orthogonal to the X-ray beam transmitted from the X-ray generation system 404 to the X-ray collection system 406. As described above, the medical system 100 typically has many degrees of freedom that can be adjusted. The degrees of freedom can be provided by the imaging system 402, the table T, another device adjusting the relative positioning and orientation of imaging system 402 and patient P, or any combination thereof. For example, in the depiction of FIG. 4, the imaging system is mobile and provides at least the following degrees of freedom: first translational DOF 408, second translational DOF 410, first rotational DOF 415, second rotational DOF 411, and third rotational DOF 407. Adjustment, or selection of values, for the degrees of freedom of the medical system 100 can affect the 2D imaging plane on which a 2D image is acquired using an imaging system 402. For simplicity, degrees of freedom of the medical system 100 that can affect the 2D imaging plane are referred to hereafter as imaging plane degrees of freedom (DOFs), regardless of whether these degrees of freedom are provided by the imaging system 402, table T, or some other device of the medical system 100. As such, a 2D imaging plane can be specified through selection of the of the imaging plane DOFs.

It is emphasized that while FIG. 4 depicts an example imaging system 402 as a mobile C-arm style imaging system capable of both CBCT (for 3D imaging) and fluoroscopy (for 2D imaging), embodiments of this disclosure are not limited to this type of imaging system 402. In general, embodiments disclosed herein can use any imaging system 402, including an imaging system 402 composed of more than one imaging device, so long as the imaging system 402 can be used to acquire images of mixed dimensionality including at least a 2D image (e.g., 2D and 3D, 2D and 2.5D, etc.).

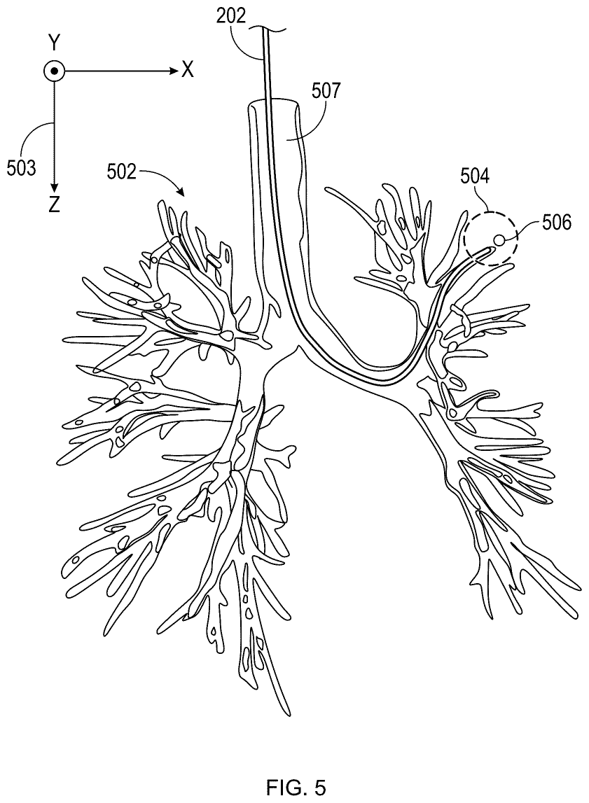

FIG. 5 depicts an example 3D model derived from an image acquired using an imaging system 402, such as the imaging system 402 depicted in FIG. 4. Specifically, FIG. 5 depicts a 3D representation (or 3D “image”) of an anatomical structure 502 (e.g., lower respiratory tract) and a flexible elongate device 202 within the anatomical structure 502. FIG. 5 further depicts a reference coordinate system 503 from which spatial information for the anatomical structure 502 and flexible elongate device 202 can be determined. In the example of FIG. 5, the anatomical structure 502 is a lower respiratory tract and the reference coordinate system 503 is a right-handed Cartesian coordinate system where the Z-axis is approximately aligned with the trachea 507, however, in practice the anatomical structure 502 or the reference coordinate system 503 need not be as shown in FIG. 5. FIG. 5 also depicts a target region 504, i.e., a region or volume proximate a target structure 506 (e.g., PPL). Generally, the target structure 506 can be identified in a 3D image. In some embodiments, the 3D image is acquired using cone beam computed tomography (CBCT) and has a limited temporal resolution of, for example, no better than 0.1 Hz. Alternatively, tomosynthesis imaging may be used to obtain quasi-3D images (so-called 2.5D images).

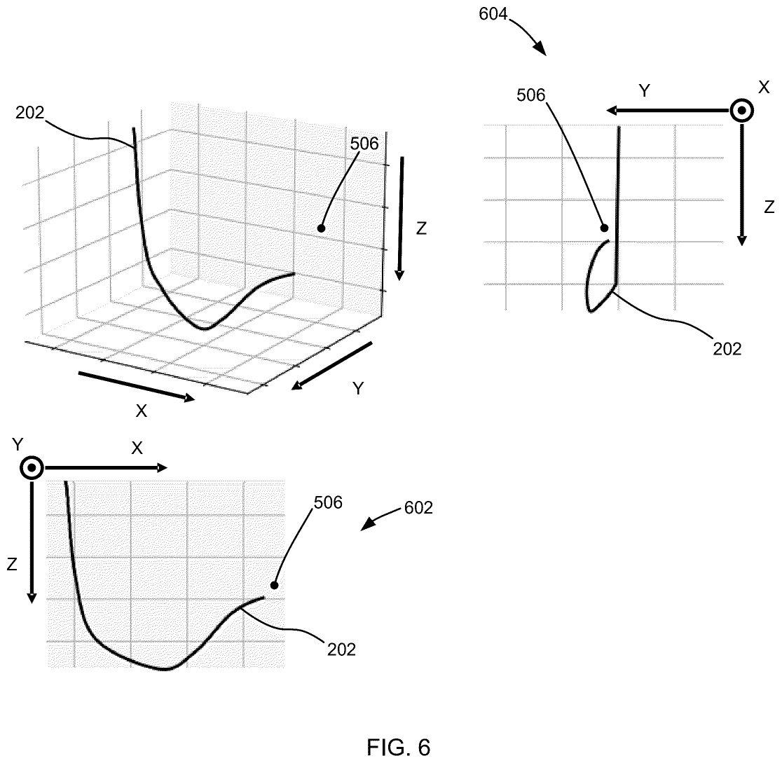

FIG. 6 depicts an alternative viewpoint of the flexible elongate device 202 shown in FIG. 5 with respect to the given reference coordinate system 503 along with a first projection 602 and a second projection 604. Specifically, the first projection 602 depicts the flexible elongate device 202 projected onto the X-Z plane and the second projection 604 depicts the flexible elongate device 202 projected onto the Y-Z plane, according to the given reference coordinate system 503. In general, a 2D image acquired using an imaging system 402 represents a projection of the imaged region onto a 2D imaging plane. As previously described, a 2D imaging plane can be selected through adjustment or selection of one or more imaging plane DOFs. It is noted that while FIG. 6 depicts projections 602, 604 that are coplanar with the X-Z plane and Y-Z plane, respectively, selection of a 2D imaging plane is not limited to these projections. In general, any 2D imaging plane can be selected so long as it can be achieved using the imaging plane DOFs provided by the medical system 100. Further, as seen in FIG. 6, the target structure 506 is depicted on the first projection 602 and the second projection 604. Often, the target structure 506 is not distinguishable in a 2D image acquired using an imaging system 402. As such, in some embodiments, one or more 3D images, where the target structure 506 is distinguishable, are acquired to ascertain the absolute and/or relative positioning of the flexible elongate device 202 and the target structure 506 and the 2D images are augmented with a graphical representation of the target structure 506 based on the 3D image. In some embodiments, 2D images are obtained using fluoroscopy imaging with a temporal resolution approximately greater than or equal to 10 Hz. Generally, 2D images can be acquired with a temporal resolution greater than their 3D image counterparts. For example, in some instances, the temporal resolution of 2D images is two orders or magnitude greater than the temporal resolution of 3D images.

Operating a medical system 100 that uses a flexible elongate device 202 (e.g., catheters or endoscopes)) may involve performing a medical operation (e.g., a biopsy) at a target region 504 using a tool 226 (e.g., a biopsy needle) inserted through the flexible elongate device 202. Performing the medical operation may involve a targeting operation that includes aiming the flexible elongate device 202 towards the target structure 506. Aiming of the flexible elongate device 202 may involve adjusting an articulation of an articulable portion of the flexible elongate device 202, and/or insertion/retraction movement of the flexible elongate device 202.

Embodiments of the disclosure use imaging feedback to control the flexible elongate device 202 when performing the targeting operation. More specifically, embodiments of the disclosure synergistically use 3D images that include a 3D representation of the flexible elongate device and the target structure 506 obtainable at a relatively low temporal frequency and 2D images that are limited to a 2D representation of the flexible elongate device 202, obtainable at a relatively high temporal frequency. In some embodiments, the availability of the combination of 3D and 2D images facilitates the targeting operation by providing a full 3D context based on the 3D images and relatively frequent updating based on the 2D images.

In some embodiments, a 3D image is acquired, and the position of the flexible elongate device 202 and the target structure 506 are identified in the resulting 3D image. Pre-operative images can be used to identify the target structure 506, construct a model of the procedural site, and register the 3D image. Accordingly, based on the 3D image, a relative position of the flexible elongate device 202 (in particular the articulable portion within or proximate a target region 504) and the target structure 506 is known, which may be used to determine a first movement of the flexible elongate device 202 towards the target site. In some embodiments, the 3D image is acquired using cone beam computed tomography (CBCT) or other 3D imaging modality and may have limited temporal resolution of, for example, no better than 0.1 Hz. Alternatively, 2.5D scans may be obtained using tomosynthesis imaging.

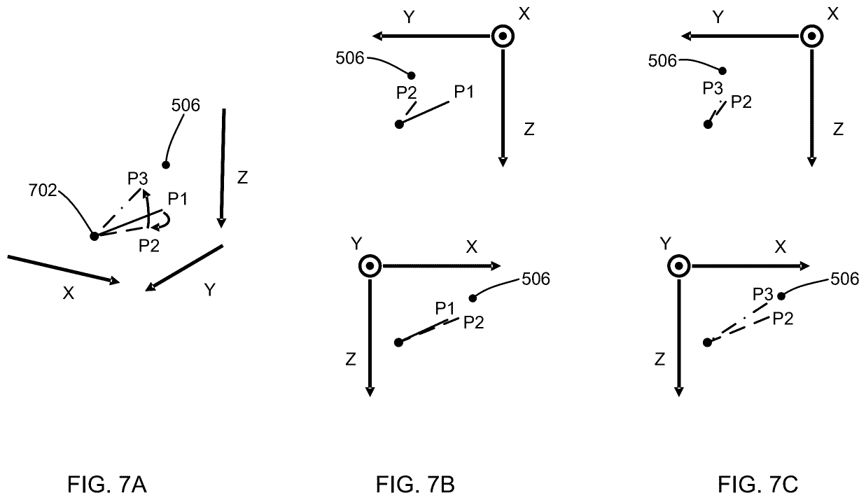

The 2D images may be obtained using fluoroscopy imaging. In general, the 2D images have a considerably higher temporal resolution than the 3D images (e.g., two orders of magnitude greater). In accordance with some embodiments, a 2D image is obtained in a 2D imaging plane selected to best visualize the current articulation of the articulable portion of the flexible elongate device 202 and the target structure 506, which may be captured in the 2D image, augmented within the 2D image, or both. The 2D imaging plane may be defined, for example, by three points, including the distal portion (e.g., the tip) of the flexible elongate device at a first position (prior to movement of the flexible elongate device) and at a second position (expected position resulting from execution of movement towards the target structure), and a point on the target structure. Subsequently, in some embodiments, a second movement that further minimizes the distance (and/or optimizes an orientation) between the distal portion of the flexible elongate device 202 and the target structure 506 (based on the 2D image) is determined, followed by an execution of the second movement. The second movement may be determined in the same 2D imaging plane as the first movement, or in a different 2D imaging plane that best visualizes the current articulation of the articulable portion. The obtaining of a 2D image, augmentation of the 2D image with a graphical representation of the target structure 506, followed by the movement of the flexible elongate device 202 may be repeated, e.g., in a loop, until the distal portion of the flexible elongate device 202 is positioned to enable execution of the medical operation, for example based on an orientation of the end effector relative to the target site (e.g., oriented towards the target site) and/or a distance between the end effector and the target site. An example is subsequently provided with reference to FIGS. 7A, 7B, and 7C.

FIGS. 7A, 7B, and 7C depict an example targeting operation, in accordance with some embodiments. FIG. 7A depicts two sequential movements of a distal portion of a flexible elongate device 202; for example, the portion of the flexible elongate device 202 in a target region 504. For simplicity, FIGS. 7A, 7B, and 7C depict movement of the distal portion of the flexible elongate device 202 from a single point of articulation 702, however, it is understood that in practice articulation of the flexible elongate device 202, or a portion of the flexible elongate device 202, may not originate from a single point. In particular, FIG. 7A depicts a 3D representation of a first movement of the distal portion of the flexible elongate device 202 from a first position P1 (distal portion identified by solid line extending from the point of articulation 702 to P1) to a second position P2 (distal portion identified by dashed line extending from the point of articulation 702 to P2) and a second movement from the second position P2 to a third position P3 (distal portion identified by dash-dotted line extending from the point of articulation 702 to P3). The first movement may be in a first direction of a pitch-yaw space of the flexible elongate device 202. Similarly, the second movement may be in a second direction of the pitch-yaw space of the flexible elongate device 202. For simplicity and ease of illustration, the distal portion of the flexible elongate device 202 in the example of FIGS. 7A, 7B, and 7C is orientated such that the first movement is in the yaw DOF of the flexible elongate device 202 and occurs in a plane substantially parallel with the X-Y plane of the illustrated reference coordinate system 503. Similarly, the second movement is in the pitch DOF of the flexible elongate device 202 and occurs in a plane substantially parallel with the X-Z plane.

FIG. 7B depicts the first movement of the distal portion of the flexible elongate device 202 from the first position P1 to the second position P2 projected onto the Y-Z plane and X-Z plane. FIG. 7C depicts the second movement of the of the distal portion of the flexible elongate device 202 from the second position P2 to the third position P3 projected onto the Y-Z plane and X-Z plane.

As seen in FIG. 7B, the first movement is better visualized in the projection onto the Y-Z plane than the projection on the X-Z plane. Similarly, as seen in FIG. 7C, the second movement is better visualized in the projection onto the X-Z plane than the projection on the Y-Z plane. Thus, if limited to these two 2D imaging planes (i.e., the Y-Z plane and the X-Z plane) 2D images taken in the Y-Z plane should be used to monitor and/or control execution of the first movement and 2D images taken in the X-Z plane should be used to monitor and/or control execution of the second movement. These 2D images best visualize the first position P1 and the second position P2 for the first movement (FIG. 7B), and the second position P2 and the third position P3 for the second movement (FIG. 7C). While not illustrated, these 2D images may also show the target structure (either captured in the 2D images, or added to the 2D images by augmentation). It is emphasized that the projections in FIGS. 7A and 7B are illustrated for their simplicity. In practice, 2D imaging planes selected to monitor and/or control execution of movements of the flexible elongate device are unlikely to be aligned with any of the axes of the medical system 100. In practice, any 2D imaging plane can be selected to visualize a given movement subject only to constraints potentially imposed by the imaging plane DOFs provided by the medical system 100, as previously discussed. In some embodiments, a 2D image is obtained in a 2D imaging plane selected, within any constraints imposed by the imaging plane DOFs, to best visualize the current articulation of the articulable portion of the flexible elongate device 202. Thus, the 2D imaging plane can be selected for each expected movement. That is, a first 2D imaging plane can be selected through corresponding adjustment of the imaging plane DOFs to visualize an expected first movement and a second 2D imaging plane can be selected through corresponding adjustment of the imaging plane DOFs to visualize an expected second movement, where the first and second 2D imaging plane may be but need not be the same. Further, while FIGS. 7A, 7B, and 7C depict two movements (namely, the first movement and the second movement), embodiments disclosed herein are not limited to two movements. In general, one or more movements can be executed in a sequence to achieve a desired result (e.g., aiming the distal portion of the flexible elongate device 202 at a target structure 506, for example, by articulating the articulable portion of the flexible elongate device 202 to reduce a distance between the distal end of the flexible elongate device 202 and the target structure 506) and a 2D imaging plane can be determined and selected for each movement.

While FIGS. 7A, 7B, and 7C illustrate an example targeting operation, a similar sequence of movements can be executed and visualized using one or more 2D imaging planes, to perform a navigating operation. In general, a navigating operation will further include movements of the flexible elongate device 202 other than articulation of a distal portion of the flexible elongate device 202 as well as bulk displacement of the flexible elongate device 202, for example, along the insertion axis. That is, in some embodiments, the method as described above may also be used for navigating of the flexible elongate device 202 towards the target structure 506, where navigating may involve significant movement along the insertion degree of freedom, in addition to movement along the articulation degrees of freedom, e.g., to follow a passage (such as an airway or other anatomical passageway).

A benefit of embodiments disclosed herein is that, upon acquiring an initial 3D image, subsequent movements of the flexible elongate device 202, whether for navigating or for targeting, can be executed, visualized, validated, and monitored using only 2D images with real-time temporal resolution (e.g., greater than 10 Hz).

An additional 3D image can be acquired under certain circumstances, e.g., triggered by an operation context. For example, an additional 3D image can be acquired when the combination of movements that have been performed exceed a certain threshold distance or angle, when there is a mismatch of the representation of the flexible elongate device 202 in the 2D image and a sensed configuration of the flexible elongate device (e.g., using a shape sensor or approximated given the historical accumulation of movements), when there is a mismatch of the representation of the flexible elongate device in the 2D image and the commanded articulation signals (suggesting out-of-plane movement), etc., as further discussed below.

The movements of the flexible elongate device 202, in some embodiments, may be under the control of an operator O of the flexible elongate device 202. In one example, a movement of the flexible elongate device 202 is performed as instructed by the operator O although limited to the trajectory of the movement as determined, e.g., based on a 2D image. Such a movement may be performed in the determined and/or selected 2D imaging plane, and a user input device operated by the operator O may be limited to accepting inputs in that plane only. To improve accuracy and reliability of the targeting operation, additional limitations may be imposed on the movement determined based on a 2D image. For example, the maximum force/torque, amplitude, and/or speed may be limited.

In some embodiments, movement of the flexible elongate device 202 can be implemented automatically (i.e., without operator O input) by the control system 112. In some embodiments, a hybrid of automated actions and operator O control is used when controlling the medical instrument 104 for navigating and/or targeting. For example, operator O may supervise automated control operations, select automated control operations, approve of proposed automated control operations, or any combination thereof. In accordance with some embodiments, and regardless of the nature of control (e.g., fully automated, operator controlled, etc.), navigating and targeting assistance is provided through, at least, the acquisition of real-time (or near real-time) 2D images of the procedure site, where the 2D images are acquired according to a determined 2D imaging plane that optimizes visualization of the movement (navigating or targeting) of the flexible elongate device 202.

In some embodiments, the medical instrument 104 is configured with a shape sensor as previously described. While not necessary to implement the methods described herein, when present, a shape sensor can be used for validation and cross-checking of the movements of the flexible elongate device 202 as visualized and measured using one or more 2D images. Further, use of the shape sensor, or other shape sensing mechanism (e.g., culmination of historical movement data), can be used to produce an uncertainty estimate and/or determine a confidence interval with respect to the position and orientation of the flexible elongate device 202 and its movements as viewed in the 2D images.

The selection of imaging planes for the control and or monitoring of movement of the flexible elongate device as described may be used in conjunction with the medical system 100 with the manipulator assembly 102 configured to drive the flexible elongate device 202 and the control system 112 coupled to the manipulator assembly. In some embodiments, the control system 112 is configured to receive a three-dimensional (3D) image of the flexible elongate device 202 (e.g., the distal portion of the flexible elongate device) and a target structure 506 and determine, based on the 3D image, a two-dimensional (2D) imaging plane for viewing movement of the flexible elongate device 202 from a first position captured in the 3D image to a second position. For example, in some implementations, the second position points toward the target structure 506 (e.g., a targeting operation). In another example, the second position positions and/or orients the flexible elongate device 202 to be closer to the target structure 506 (e.g., navigating toward a target region 504). An indication of the determined 2D imaging plane may be provided to the user in a user interface. The indication may instruct the user to reconfigure the imaging system 402 to capture one or more 2D images in the 2D imaging plane. The user may then reconfigure the imaging system as instructed. Alternatively, the reconfiguration may be automated. The control system 112 is further configured to receive one or more 2D images in the 2D imaging plane captured over time to visualize, monitor, track, or any combination thereof, the flexible elongate device 202 as it moves from the first position to the second position. Additionally, the control system 112 is configured to control the manipulator assembly 102 to move the flexible elongate device from the first position to the second position based on the 2D images (e.g., articulate and articulable portion of the flexible elongate device 202). The movement of the flexible elongated device may be driven by user input (i.e., the use steering the flexible elongate device and/or by automatic or semiautomatic algorithms.

In some embodiments, the control system 112 controls the manipulator assembly 102 to move the flexible elongate device 202 from the first position to the second position using a feedback control based on the 2D images. In other words, based on the availability of the 2D images, the control of the movement of the flexible elongate device 202 may be performed closed-loop, either manually by a user providing control inputs based on visual feedback obtained from the 2D images, or automatically by an algorithm. As an example, in a targeting operation, the feedback may be implemented as follows. In a first 2D image of the 2D images, a first spatial error between the distal portion of the flexible elongate device 202 and the target structure 506 is determined (either by the user or by the algorithm). Then, a control command that reduces the first spatial error when controlling the manipulator assembly 102 to move the distal portion of the flexible elongate device 202 is determined (either by the user or by the algorithm). The control command can be implemented to move (e.g., articulate an articulable portion) the distal portion of the flexible elongate device 202. Then, a second 2D image of the 2D images can be used to determine a second spatial error between the distal portion of the flexible elongate device 202 and the target structure 506. Based on the second spatial error, another control command that reduces the second spatial error when controlling the manipulator assembly 102 to move the distal portion of the flexible elongate device 202 can be determined. Similarly, the other control command can be implemented to move the distal portion of the flexible elongate device 202 to reduce the second spatial error. This process can be repeated in a loop as required to aim the distal portion of the flexible elongate device 202 at the target structure 506 and/or reduce a determined spatial error below a predefined threshold.

As discussed above, various imaging systems 402 can be used with the methods of this disclosure as long as the employed imaging system 402 (including sets of more than one imaging device) can acquire images of mixed dimensionality including at least as 2D image. For example, 3D images can be generated using cone beam computed tomography (CBCT), tomosynthesis (which may be 2.5D), ultrasound, or the like. Similarly, 2D images can be generated using fluoroscopy, ultrasound, or another technique for generating 2D images.

In some embodiments, the control system 112, in addition to determining an optimal 2D imaging plane based on a proposed, expected, or desired movement of the flexible elongate device 202 given 3D data from a 3D image, can generate an indication of the optimal 2D imaging plane. For example, the 2D imaging plane can be augmented onto a 3D image or otherwise displayed in a reference coordinate system. In other examples, the indication of the 2D imaging plane is provided to operator O through a user interface (e.g., master assembly 106, display system 110).

In some embodiments, the control system 112 is further configured to confirm a configuration of the imaging system 402 for capturing the 2D images by verifying the 2D imaging plane using a validation of at least one of the 2D images against the 3D image.

As discussed above, control of the manipulator assembly 102 to move the instrument 104, or, more specifically, the flexible elongate device 202, can be manual (i.e., fully user-controlled), semi-autonomous (e.g., user validated or monitored), or fully autonomous. In some embodiments, an operator O (or user) controls the manipulator assembly 102 to move the flexible elongate device 202 from the first position to the second position. However, movement of the flexible elongate device (e.g., articulation of an articulable portion) may be constrained such that the movement resides in a determined 2D imaging plane. The 2D imaging plane may be determined as the 2D imaging plane that best visualizes the intended movement of the flexible elongate device 202, where the intended movement is itself determined according to some metric (e.g., reducing the spatial error between a distal portion of the flexible elongate device 202 and a target structure 506). In some embodiments, the constraint of the movement of the flexible elongate device 202 is enforced regardless of a user control input to move the flexible elongate device 202 in some other direction.

In some embodiments, control of the manipulator assembly 102 to move the instrument 104 (or, more specifically, the flexible elongate device 202), whether the control is completely user-directed, semi-autonomous, or fully autonomous, is further constrained or limited. For example, control and movement of the flexible elongate device from a first position to a second position can include the application of one or more of the following limits: an amplitude limit; a torque limit; and a speed limit. In some embodiments, constraints or limits imposed on the movement or control of the instrument (or, more specifically, the flexible elongate device 202) are based on an operation context. The operation context may specify, among other things, a type of procedure being performed, as state of a current procedure, and a use of a medical tool 226. For example, an operation context can indicate that the medical tool 226 conveyed to a target region 504 by the flexible elongate device 202 is a biopsy needle. In this case, the operation context may further indicate whether the biopsy needle is in an extended or non-extended state. In some embodiments, a volumetric constraint is determined based on one or more of the 3D image(s), 2D images, and the operation context. The volumetric constraint may block movement of the instrument 104 (e.g., the distal portion of a flexible elongate device 202) to reside within a volume define by a boundary surface and block movements of the instrument 104 to positions that reside outside of the volume. As an example, given an operation context of an extended biopsy needle, all movement of the instrument 104 can be blocked (or limited) except for an insertion (and retraction) movement of the medical tool 226 (i.e., the biopsy needle).

In some embodiments, an operation context can trigger one or more behaviors of the medical system 100. For example, a triggered behavior can include the acquisition of an additional 3D image, and based on the additional 3D image, determine an update to the 2D imaging plane for viewing movement of the flexible elongate device 202. Examples of an operation context that may trigger a behavior or action (e.g., acquisition of an additional 3D image) can include, but are not limited to: a divergence of an observed movement (observed in 2D images) from an expected movement (e.g., commanded movement from a known first position to a specified second position), where the divergence exceeds a predefined threshold (where the threshold may also be dependent on the operation context such as the type of procedure being performed); patient P motion; and a mismatch between a representation of the flexible elongate device 202 in at least one of the 2D images and a sensed configuration of the flexible elongate device 202, the mismatch suggesting out-of-plane movement. In this latter case, the sensed configuration can be acquired using a shape sensor, if applicable. Divergence of an observed movement from an expected movement can be measured in a variety of ways, including, determining a Euclidean distance between the observed second position and the expected second position or liming movement of flexible elongate device (e.g., the distal portion) to a given path distance, Euclidean distance, angle (measured relative to some point of articulation), or combination thereof.

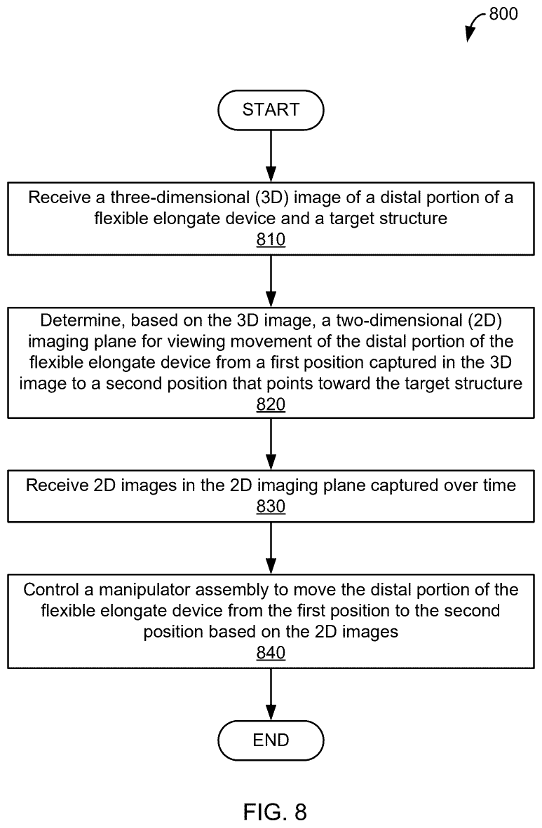

FIG. 8 depicts a method 800 for executing a targeting operation with feedback from one or more 2D images, the one or more 2D images acquired according to one or more determined 2D imaging planes, the one or more 2D imaging planes each selected to optimally visualize a corresponding or expected movement of the flexible elongate device 202. The method may be implemented using instructions stored on a non-transitory medium that may be executed by a computing system, e.g., the computing system 120.

While the various blocks in FIG. 8 are presented and described sequentially, some or all of the blocks may be executed in different orders, may be combined or omitted, and some or all of the blocks may be executed in parallel. Furthermore, the blocks may be performed actively or passively.

Turning to FIG. 8, in block 810, a 3D image of a distal portion of a flexible elongate device 202 and a target structure 506 is received by a control system 112 (or, more generally, the computing system 120) of a medical system 100 including a manipulator assembly 102 for controlling or manipulating, at least, the flexible elongate device 202. The 3D image is acquired using an imaging system 402 of the medical system 100. In some implementations, acquisition of the 3D image may require 10 or more seconds. Thus, in these implementations, an acquired sequence of 3D images may be said to have a temporal resolution no greater than 0.1 Hz (or, more simply, the temporal resolution of 3D images is no greater than 0.1 Hz). The 3D image can be acquired using cone beam computed tomography (CBCT), ultrasound, or some other 3D imaging technique.

In block 820, based on the 3D image, a 2D imaging plane for viewing movement of the distal portion of the flexible elongate device 202 from a first position to a second position is determined. The first position is captured (i.e., known, visible, and spatially referenced with respect to the surrounding anatomical structure) in the 3D image. The second position points toward the target structure 506 or moves (e.g., articulates) the distal portion of the flexible elongate device 202 in such a manner as to reduce the distance between the distal end of the flexible elongate device 202 and the target structure 506 (e.g., a movement in a sequency of movements that, when executed, point the distal portion of the flexible elongate device 202 at the target structure 506). The determined 2D imaging plane is the optimal plane for viewing the movement of the distal portion of the flexible elongate device 202 subject to any constraints imposed by the imaging plane DOFs of the medical system 100 (e.g., as previously described in reference to the example of FIGS. 7A-7C. An indication of the determined 2D imaging plane may further be provided to the user in a user interface (e.g., master assembly 106, display system 110). The indication may include instructions for how to reconfigure the imaging system 402 to capture one or more 2D images in the 2D imaging plane. The user may then reconfigure the imaging system as instructed. Alternatively, the reconfiguration may be automated.

While not identified as a separate operation in the method 800, the imaging system is then reconfigured as needed to capture 2D images in the 2D imaging plane determined in block 820. The reconfiguration may be performed either manually by the user, or automatically.

In block 830, 2D images are received in the 2D imaging plane captured over time to visualize the movement of the distal portion of the flexible elongate device 202 from the first position to the second position. The temporal resolution of the 2D images is greater than the temporal resolution of the 3D images. In some implementations, acquisition of a 2D image may be performed in less than one-tenth of a second. Thus, in these implementations, an acquired sequence of 2D images (i.e., 2D images captured over time) may be said to have a temporal resolution of 10 Hz or greater (or, more simply, the temporal resolution of 2D images is greater than 10 Hz). In some embodiments, the temporal resolution of the 2D images is sufficient for rea-time use.

In block 840, the manipulator assembly 102 is controlled to move the distal portion of the flexible elongate device 202 from the first position to the second position based on the 2D images. That is, the 2D images are used as real-time feedback to visualize, validate, and ensure the movement of the distal portion of the flexible elongate device 202 from the first position to the second position. Upon validation, subsequent movements of the distal portion of the flexible elongate device 202 (e.g., from the second position to a third position) can be implemented using feedback based on 2D images acquired according to another 2D imaging plane determined to view the subsequent movement(s). This process may be repeated, as necessary, to achieve the desired position and orientation of the flexible elongate device 202 without acquisition of another 3D image unless triggered by an operation context.

Embodiments of the disclosure have various benefits. Because 2D images are obtainable at a temporal resolution greater than their 3D (or 2.5D) image counterparts, embodiments disclosed herein allow for the real-time movement and feedback control of an instrument (e.g., in a targeting or navigating operation) based on 2D images captured over time in one or more determined 2D imaging planes. The 2D imaging planes can be determined and adjusted to (i.e., though adjustment of imaging plane DOFs) automatically. Thus, real-time positioning of an instrument 104 in an anatomical structure can be determined and validated without a dedicated shape sensor or continuous acquisition of 3D images (subject to the 3D image temporal resolution and excepting any triggers based on operation context).

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical system comprising:
a manipulator assembly configured to drive a flexible elongate device; and
a control system coupled to the manipulator assembly, the control system configured to:
receive a three-dimensional (3D) image of a distal portion of the flexible elongate device and a target structure;
determine, based on the 3D image, a two-dimensional (2D) imaging plane for viewing movement of the distal portion of the flexible elongate device from a first position captured in the 3D image to a second position that points toward the target structure;
receive 2D images in the 2D imaging plane captured over time; and
control the manipulator assembly to move the distal portion of the flexible elongate device from the first position to the second position based on the 2D images.

2. The medical system of claim 1, wherein the control system controls the manipulator assembly to move the distal portion of the flexible elongate device from the first position to the second position using a feedback control based on the 2D images.

3. The medical system of claim 2, wherein using the feedback control based on the 2D images comprises:
in a first 2D image of the 2D images, determining a first spatial error between the distal portion of the flexible elongate device and the target structure, and
determining a control command that reduces the first spatial error when controlling the manipulator assembly to move the distal portion.

4. The medical system of claim 3, wherein using the feedback control based on the 2D images further comprises:
in a second 2D image of the 2D images, determining a second spatial error between the distal portion of the flexible elongate device and the target structure, and
determining a control command that reduces the second spatial error when controlling the manipulator assembly to move the distal portion.

5. The medical system of claim 1, wherein the 3D image is generated using one selected from the group consisting of:
cone beam computed tomography (CBCT), and
tomosynthesis.

6. The medical system of claim 1, wherein at least one of the 3D image and 2D images are generated using ultrasound.

7. The medical system of claim 1, wherein the 2D images are generated using fluoroscopy.

8. The medical system of claim 1, wherein the 2D image plane is further determined such that the 2D images include the target structure.

9. The medical system of claim 1, wherein the control system is further configured to:
augment the 2D images with a graphical representation of the target structure.

10. The medical system of claim 1, wherein the control system is further configured to:
generate an indication of the 2D imaging plane, and
provide the indication to a user interface.

11. The medical system of claim 10, wherein the control system is further configured to confirm a configuration of a 2D imaging device for capturing the 2D images by verifying the 2D imaging plane using a validation of at least one of the 2D images against the 3D image.

12. The medical system of claim 1, wherein the control system is further configured to:
provide instructions for reconfiguring an imaging device to enable capturing of the 2D images in the 2D imaging plane,
wherein the instructions are provided to one selected from the group consisting of a user interface and the imaging device.

13. The medical system of claim 1, wherein controlling the manipulator assembly to move the distal portion of the flexible elongate device from the first position to the second position comprises:
constraining the movement of the distal portion based on a user control input.

14. The medical system of claim 1, wherein controlling the manipulator assembly to move the distal portion of the flexible elongate device from the first position to the second position comprises:
constraining the movement of the distal portion to a direction from the first position to the second position, regardless of a user control input.

15. The medical system of claim 1, wherein controlling the manipulator assembly to move the distal portion of the flexible elongate device from the first position to the second position comprises at least one selected from the group consisting of applying an amplitude limit, applying a torque limit, and applying a speed limit to the movement of the distal portion.

16. The medical system of claim 1, wherein the control system is further configured to:
determine a first operation context, and based on the first operation context, allow the control of the manipulator assembly to move the distal portion of the flexible elongate device from the first position to the second position based on the 2D images.

17. The medical system of claim 16, wherein the first operation context comprises a non-extended biopsy needle.

18. The medical system of claim 16, wherein the control system is further configured to:
determine a second operation context, and based on the second operation context, block a control of the manipulator assembly to move the distal portion of the flexible elongate device from the second position to a third position based on the 2D images.

19. The medical system of claim 18, wherein the second operation context comprises one selected from the group consisting of:
an extended biopsy needle and
an insertion movement of the flexible elongate device required by the movement of the distal portion of the flexible elongate device from the second position to the third position.

20. The medical system of claim 1, wherein the movement of the distal portion of the flexible elongate device from the first position to the second position comprises at least one selected from the group consisting of:
an articulation of the articulable portion and
an articulation of the articulable portion in combination with a retraction movement of the flexible elongate device.

21. The medical system of claim 1, wherein the control system is further configured to:
receive an additional 3D image, and
determine, based on the additional 3D image, an updated 2D imaging plane for viewing movement of the distal portion of the flexible elongate device from the second position captured in the 3D image to a third position that points toward the target structure.

22. The medical system of claim 21, wherein the control system is further configured to:
determine an operation context, and wherein the receiving of the additional 3D image is performed based on the operation context.

23. The medical system of claim 22, wherein the operation context comprises at least one selected from the group consisting of:
a difference in a configuration of the flexible elongate device after execution of the movement of the distal portion of the flexible elongate device from the first position to the second position, and wherein the difference exceeds a threshold value,
a patient motion,
a mismatch between a representation of the flexible elongate device in at least one of the 2D images and a sensed configuration of the flexible elongate device, and
a mismatch between an articulation of an articulable body portion in a representation of the flexible elongate device in at least one of the 2D images and a commanded articulation.
